(19) United States
(12) Patent Application Publication
(10) Pub. No.: US 2016/0278844 A1
(43) Pub. Date: Sep. 29, 2016

(54) MICROSLOTTED ELECTROSURGICAL DEVICE WITH SMOKE EVACUATION

(71) Applicant: Medtronic Advanced Energy LLC, Minneapolis, MN (US)

(72) Inventors: Nathan ZAMARRIPA, Kittery Point, ME (US); Brian M. CONLEY, South Berwick, ME (US); Tarquinio A. BRUNO, Hampton, NH (US)

(21) Appl. No.: 14/670,032

(22) Filed: Mar. 26, 2015

(51) Int. Cl. A61B 18/14

(52) U.S. Cl. CPC A61B 18/14; A61B 18/1482; A61B 2018/00589

(57) ABSTRACT

An electrosurgical device including an elongate body including a rigid proximal portion, a distal portion, and defining a lumen there though. A fluid delivery tube is disposed within the lumen. A first electrode coupled to the distal end of the elongate body is included, the first electrode is configured to ablate tissue with radiofrequency energy. The distal portion includes a plurality of slots in fluid communication with the lumen. The plurality of slots are spaced a longitudinal distance proximal from the first electrode. The plurality of slots are configured to aspirate gas released from tissue ablated with the first electrode. The plurality of slots are further configured to provide malleability to the distal portion.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to electrosurgical devices and systems, and in particular, an electrosurgical device having an integral and malleable suction feature.

### BACKGROUND OF THE INVENTION

An electrosurgical device is a medical device configured to treat a patient's tissue with radiofrequency energy to dissect and/or to coagulate a target tissue region. Such electrosurgical devices typically include a hand piece with one or more electrodes at the distal end of the hand piece in electrical communication with a radiofrequency generator. Some of those electrosurgical devices also include the capability to irrigate the target tissue region with saline. However, when irrigation is utilized during application of radiofrequency energy, or when radiofrequency energy is used to treat a target tissue region having water, large amounts smoke and/or steam may be produced that can hinder the surgeons ability to treat the target tissue region by blocking a clear view of the treatment region and by providing unwanted materials at the treatment site.

Accordingly, smoke evacuators have been developed to suction smoke and electrosurgical byproducts from the surgical site, such as charred tissue. A smoke evacuator typically includes a separate suction tube in fluid communication with a vacuum source. Surgeons often must stop the procedure as smoke builds up around the surgical site to suction to the smoke and the additional tube is often cumbersome and may interfere with the procedure. For example, a surgical staff person other than the surgeon must usually hold and manipulate the end of the suction tube while the surgeon manipulates the electrosurgical device during the medical procedure. Thus, treatment times and costs of such electrosurgical procedures are often increased.

### SUMMARY

The present invention advantageously provides an electrosurgical device including an elongate body including a rigid proximal portion, a distal portion, and defining a lumen there though. A fluid delivery tube is disposed within the lumen. A first electrode coupled to the distal end of the elongate body is included, the first electrode is configured to ablate tissue with radiofrequency energy. The distal portion includes a plurality of slots in fluid communication with the lumen. The plurality of slots are spaced a longitudinal distance proximal from the first electrode. The plurality of slots are configured to aspirate gas released from tissue ablated with the first electrode. The plurality of slots are further configured to provide malleability to the distal portion.

In another embodiment, the electrosurgical device includes an elongate body including a rigid proximal portion, a distal portion, and defining a lumen there though. A fluid delivery tube is disposed within the lumen. A first electrode coupled to the distal end of the elongate body is included, the first electrode is configured to ablate tissue with radiofrequency energy. The distal portion includes a plurality of slotted sections circumferentially disposed about the elongate body in fluid communication with the lumen. The plurality of slotted sections are spaced a longitudinal distance proximal from the first electrode. The plurality of slotted sections are configured to aspirate gas released from tissue ablated with the first electrode. The plurality of slotted sections are further configured to provide malleability to the distal portion in all directions. The distal portion defines a plurality of unslotted sections, each unslotted section is disposed between adjacent slotted sections, the unslotted sections provide stiffness to the distal portion.

In yet another embodiment, the electrosurgical device includes an elongate body including a rigid proximal portion, a distal portion, and defining a lumen there though. A fluid delivery tube is disposed within the lumen. A first electrode and a second electrode are coupled to the distal end of the elongate body. The first electrode and the second electrode define a substantially co-planar surface. The first electrode and the second electrode are configured to ablate tissue with radiofrequency energy. The distal portion includes a plurality of slotted sections circumferentially disposed about the elongate body in fluid communication with the lumen. The plurality of slotted sections are spaced a longitudinal distance proximal from the first electrode. The plurality of slotted sections are configured to aspirate gas released from tissue ablated with the first electrode and the second electrode. The plurality of slotted sections are further configured to provide malleability to the distal portion in all directions. Each slot in the plurality of slotted sections defines a width between 0.01 inches and 0.03 inches. Each of the plurality of slotted sections includes between twenty and thirty slots. Each slot in the plurality of slotted sections is offset from an adjacent one of the plurality of slots by an angle between 10 degrees and 30 degrees. The distal portion defines a plurality of unslotted sections, each unslotted section is disposed between adjacent slotted sections, the unslotted sections provide stiffness to the distal portion.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is front perspective view of an electrosurgical device constructed in accordance with the principles of the present application;

FIG. 2 is a side cross-sectional view of a handle portion of the electrosurgical device shown in FIG. 1;

FIG. 3 is a front perspective view of a distal portion of the electrosurgical device shown in FIG. 1;

FIG. 4 is a front view of the distal portion of the electrosurgical device shown in FIG. 3 treating tissue and aspirating smoke from the treatment site; and

FIG. 5 is a zoomed in view of a plurality of slots on the distal portion shown in FIG. 3.

### DETAILED DESCRIPTION OF THE INVENTION

As used here, relational terms, such as “first” and “second,” “over” and “under,” “front and rear,” and the like, may be used solely to distinguish one entity or element from another entity or element without necessarily requiring or implying any physical or logical relationship or order between such entities or elements.

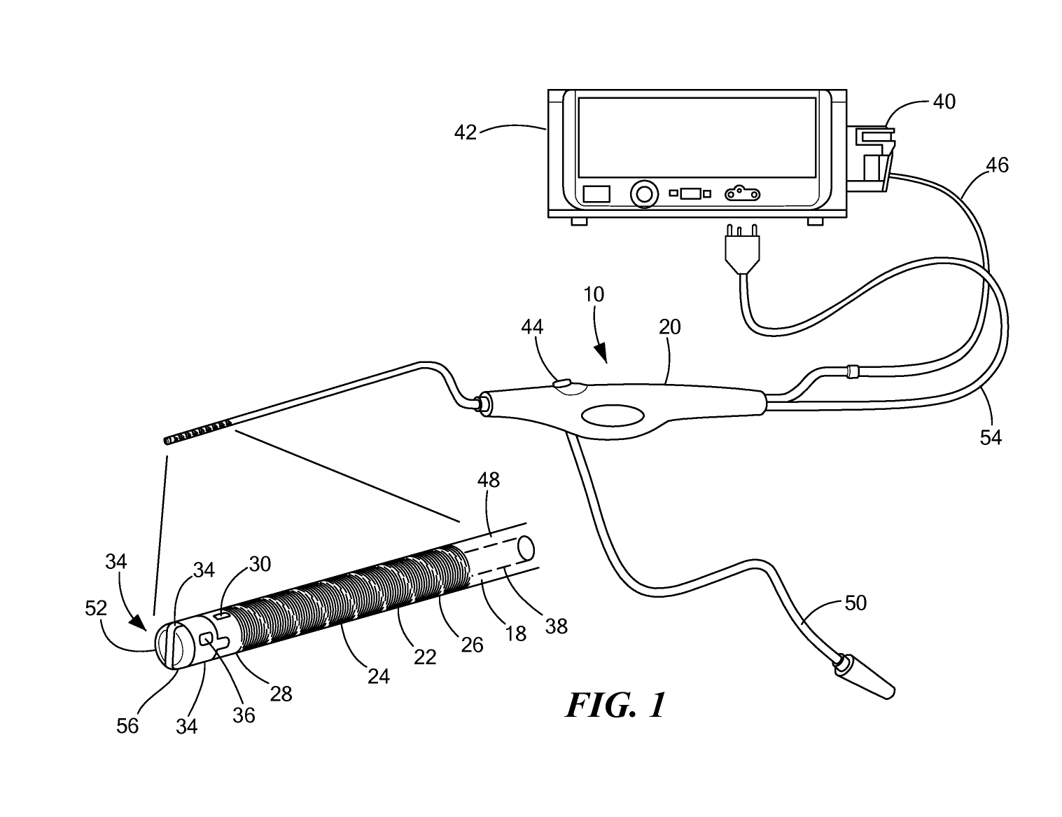

Referring now to the drawings in which like reference designators refer to like elements, there is shown in FIG. 1 an electrosurgical device constructed in accordance with the present application and designated generally as “10.” The device 10 may include an elongate body 12 defining a proximal portion 14, a distal portion 16, and a lumen 18 there through. The proximal portion 14 may be rigid and may be composed of, for example, stainless steel, titanium, or other metals or metal alloys, and may be coupled to a handle 20 at its proximal end. In one configuration, the proximal portion 14 defines a bend such that a section of the proximal portion 14 coupled to the handle 20 is off-set from a more distal section of the proximal portion 14. In other configurations, the proximal portion 14 is substantially linear in shape, however, it may define any shape as extends toward the distal portion 16.

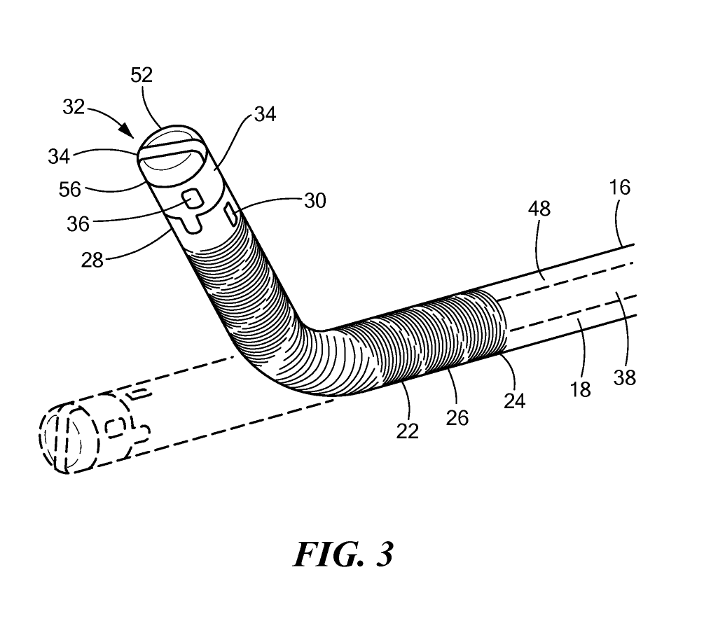

The distal portion 16 may be continuous with the proximal portion 14 and at least a portion of the distal portion 16 may define a plurality of slotted sections 22 disposed about the elongate body 12. In one configuration, the plurality of slotted sections 22 are circumferentially disposed about the distal portion 16. Each one of the plurality of slotted sections 22 may include a plurality of slots 24 in fluid communication with the lumen 18. Each of the plurality of slots 24 defines a length of approximately between 0.05 in—0.07 in or a radial length around the elongate body 12 equal to between 0.3 and 0.5 of the diameter of the elongate body 12 depending on the diameter of the elongate body 12. and may define a width of approximately 0.01 mm to 0.05 mm. In an exemplary configuration eight slotted sections 22 are included along distal portion 16 spaced a longitudinal distance apart from an adjacent section 22, each slotted section having between 20 and 30 slots 24. In other configurations, any number of slotted sections 22 may be included having any number of slots 24. The plurality of slotted sections 22 are configured to provide malleability to the distal portion 16 where the slots 24 are included. In particular, the slots 24 may be laser or machine cut into the elongate body 12, thereby reducing the strength and rigidity of the elongate body 12 in the distal portion 16 such that the distal portion 16 is malleable up to 360 degrees in all directions. In particular, the distal portion 16 may be transitionable from a substantially linear configuration, as shown in FIG. 1, to a second position, for example, the configuration shown in FIG. 3, until manipulated to a third position. To lend malleability to the distal portion, a plurality of unslotted sections 26 are disposed between adjacent slotted sections 22. The unslotted sections 26 are uncut portions of the elongate body 12 that add strength to the distal portion 16 to facilitate the malleability of the distal portion 16. In an exemplary configuration, each unslotted section 26 is approximately the same width each slot 24, however, in other configurations, the unslotted sections 26 may define a larger width which may increase the stiffness of the distal portion 16.

Continuing to refer to FIG. 1, disposed distal to the most distal slotted section 22 may be an intermediation section 28 of the elongate body 12. The intermediate section 28 may be an unslotted rigid portion of the elongate body 12 that defines one or more apertures 30 for connection of a treatment tip 32 to the distal end of the elongate body 12. In particular, the one or more apertures 30 of the intermediate section 28 may be sized to receive one or more tabs (not shown) on an electrical insulator 34 affixable to the intermediate section 28. The one or more tabs may be molded, welded, glued or otherwise affixed within the one or more apertures 30, or alternatively may releasably mate with the one or more apertures 30 such that the treatment tip may be modular with the elongate body 12. In one configuration, the affixation of the one or more tabs within the one or more apertures 30 provides an electrical connection between the elongate body 12 and the treatment tip 32. For example, disposed within the insulator 34 may be one or more conductors (not shown) that connect with one or more conductors disposed within the elongate body 12.

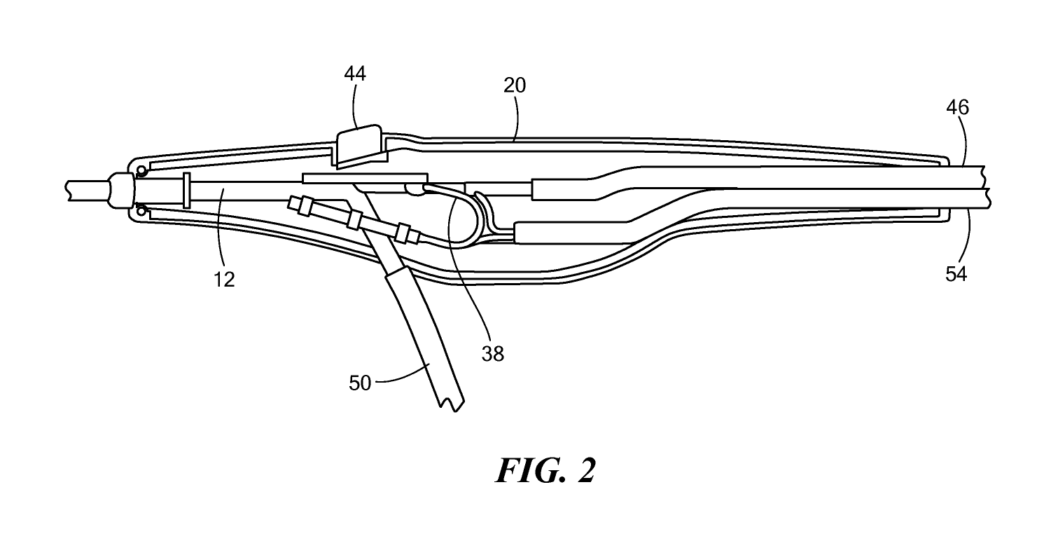

Referring now to FIGS. 1 and 2, the electrical insulator 34 may be ceramic or other electrical insulators known in the art. The electrical insulator 34 may define the same circumference to that of the intermediate section 28 and that of the elongate body 12 to provide for a uniform circumference along the length of the distal portion 16. In one configuration, the insulator 34 defines a port 36 in fluid communication with a fluid delivery tube 38 (seen in FIG. 2) disposed within the lumen 18. The port 36 may be sized to perfuse a conductive fluid, for example, saline out of the port 36 and onto a target tissue region. In one configuration, two ports 36 are disposed on opposite sides of the insulator 34, however, any number of ports 36 are contemplated. The fluid delivery tube 38 may be disposed within the lumen 18 and extend from within the handle 20 to the port 36. In an exemplary configuration, the fluid delivery tube 38 is in fluid communication with a fluid source (not shown) and a pump 40 disposed on a radiofrequency generator 42 configured to pump a fluid within the fluid delivery tube 38 toward the distal end of the device 10. For example, one or more actuators 44 may be included on the handle 20 that allow the activation of one or more features of the device 10, for example, fluid flow and radio frequency energy transmission. One of the actuators 44 may facilitate the flow of fluid from the fluid source, into the pump 40, through a fluid delivery umbilical 46 that includes a connector to connect the device 10 to the generator 42.

The fluid delivery tube 38 may be flexible and non-conductive and defines an outer diameter less than the inner diameter of the elongate body 12 such that a vacuum space 48 is defined between the fluid delivery tube 38 and the elongate body 12 within the lumen 18. The vacuum space 48 may define a pathway through which smoke, steam, and biological material may be aspirated through the plurality of slots 24 from a surgical treatment site. In particular, a vacuum umbilical 50 may be in fluid communication with the lumen 18, in particular, the vacuum space 48 between the fluid delivery tube 38 and the inner wall of the elongate body 12 and may be further connected to a vacuum (not shown), which may be integrated with the generator 42 or may be a separate unit. The vacuum may automatically suction smoke, steam, and biological material during a procedure, and substantially simultaneously perfuse saline to the treatment site. For example, the generator 42 may be configured to irrigate the treatment site with saline while at the same time or sequentially, vacuum smoke and steam generated during treatment.

Disposed at the treatment tip 32 may be at least one electrode 52 configured to transmit radio frequency energy to the treatment site. In one configuration, the at least electrode 52 defines a crescent or semi-circular shape and defines the same or similar diameter as that of the elongate body 12. In other configurations, the at least one electrode 52 may be any shape or size and may define a substantially planar surface, a blunt surface, or a sharp surface. The at least one electrode 52 may be in electrical communication with the radiofrequency generator 42 through one or more conductors (not shown) extending through the elongate body 12. When the treatment tip 32 is attached to the distal end of the elongate body 12 the at least one electrode 52 is placed in electrical communication with the one or more conductors. The handle 20 may include one or more electrical connectors 54 extending from its proximal end that connect to the radiofrequency generator 42 such that different a different voltages and power levels may be applied to the at least one electrode 52. In an exemplary configuration, the at least one electrode 52 is configured to deliver monopolar energy to the target tissue region. For example, a reference electrode (not shown) may be included as a back plate that connects to the generator 42. The voltage and power levels applied to the at least one electrode 52 may be provided to cut or coagulate tissue. In other configurations, a second electrode 56 may be included proximate the at least one electrode 52. In such a configuration, the insulator 34 may be disposed between the electrode 52 and the electrode 56, for example as shown in FIGS. 1-4. The second electrode 56 may substantially the same size and shape as the electrode 52 and may be shielded from conducting radiofrequency energy with the electrode 52. For example, the distal portion 16 may include the components disclosed in U.S. Pat. No. 8,216,233 filed Mar. 21, 2008, the entirety of which is expressly incorporated herein by reference, along with an internal evacuation passageway in fluid communication with the slots.

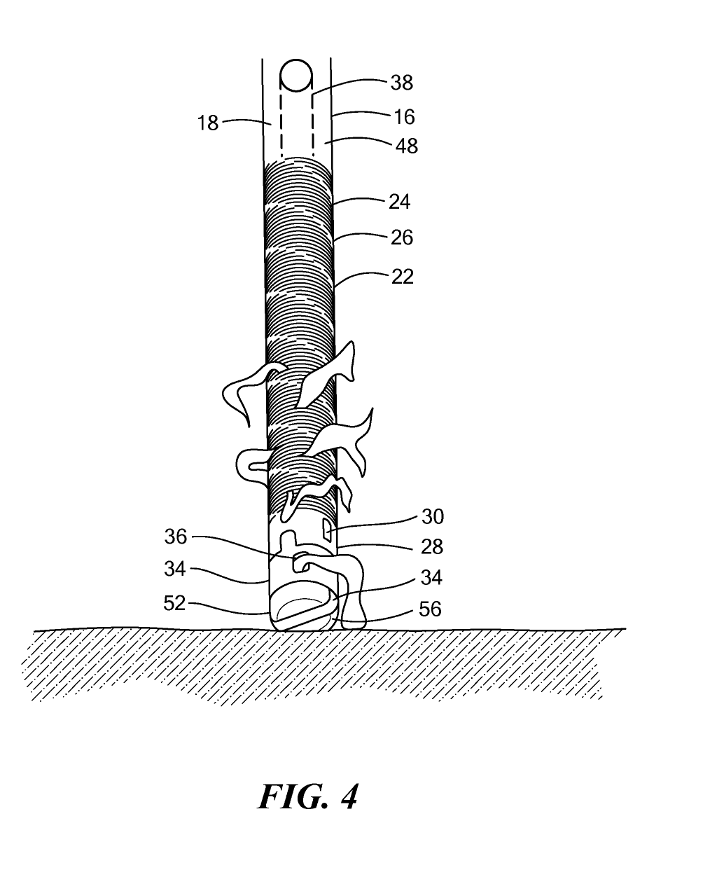

In an exemplary use of the device 10, for example as shown in FIG. 4, the treatment tip 32 may be pressed against a target tissue in a vertical orientation. In other configurations, owing the malleability of the distal portion 16, the treatment tip 32 may be disposed at angle with respect to the major longitudinal axis of the elongate body 12. The user may press the actuator 44 to provide power to the electrode 52 and second electrode 56 and/or initiate a flow of saline through the fluid delivery tube 38. The saline provides a conductive medium such that bipolar radiofrequency may flow between the electrode 52 and the second electrode 56, which may coagulate and seal the target tissue region. The saline may exit the port 36 energized with radiofrequency energy, or alternatively may be energized upon perfusion onto the target tissue. As a result of radiofrequency energy treatment from the electrode 52 and second electrode 56, the saline may begin to boil and release steam. Moreover, the water within the target tissue may boil and the destroyed tissue may release smoke. Thus, the suction device may aspirate the smoke and steam from the target tissue region through the plurality of slots 24 during the radiofrequency energy treatment without interfering with the treatment procedure or requiring an additional suction device.

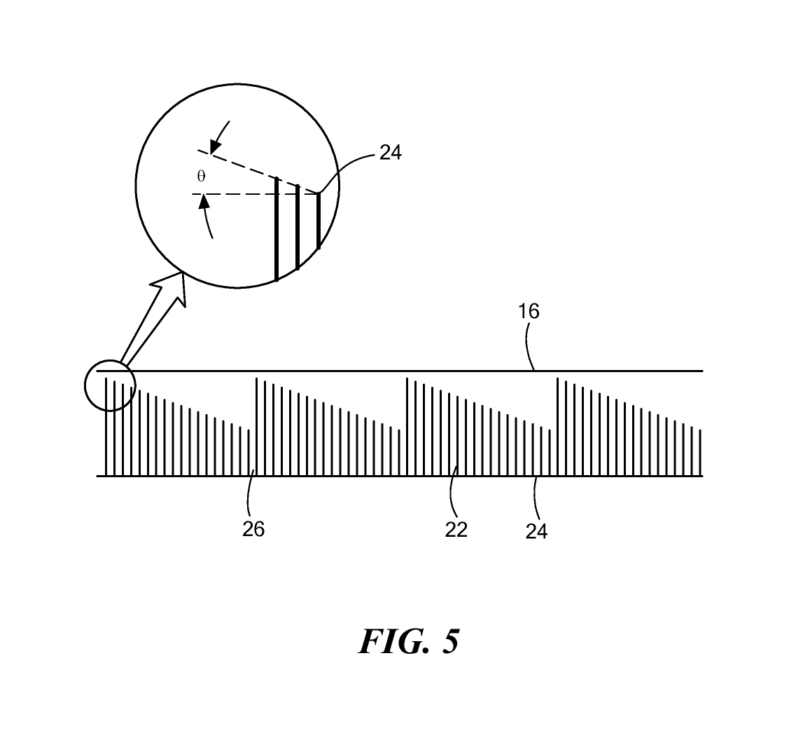

Referring now to FIG. 5, to facilitate aspiration of steam, smoke, and biological fluids from the treatment site and to facilitate the malleability of the distal portion 16, the plurality of slots 24 may be offset from an adjacent slot within the same plurality of sections 22. For example, each slot 24 may be longitudinally offset by an angle “θ,” within each of the plurality of slotted sections 22. For example, each slot may be offset from an adjacent slot by an angle of 15 degrees, such that the plurality of slots 24 in each of the plurality of slotted sections 22 winds around the circumference of the elongate body 12. In other configurations, the angle θ may range between 5 and 30 degrees. The offsetting of adjacent slots 24 provides for the malleability of the distal portion by facilitating bending of the distal portion 16 while providing stiffness such that the distal portion 16 remains in a first position until manipulated a section position.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. An electrosurgical device, comprising:
an elongate body including a rigid proximal portion, a distal portion, and defining a lumen there though,
a fluid delivery tube disposed within the lumen;
a first electrode coupled to the distal end of the elongate body, the first electrode being configured to ablate tissue with radiofrequency energy; and
the distal portion including a plurality of slots in fluid communication with the lumen, the plurality of slots being spaced a longitudinal distance proximal from the first electrode, the plurality of slots being configured to aspirate gas released from tissue ablated with the first electrode, the plurality of slots being further configured to provide malleability to the distal portion.

2. The device of claim 1, further including a port in fluid communication with the fluid delivery tube, the port being disposed distal to the plurality of slots and proximal to the first electrode, the port being configured to expel fluid from the fluid delivery tube.

3. The device of claim 2, further include an electrical insulator disposed between the first electrode and the plurality of slots, and wherein the port is disposed within the insulator.

4. The device of claim 3, further including a second electrode adjacent the first electrode, and wherein the electrical insulator is disposed between the first electrode and the second electrode, and the first electrode and the second electrode are configured to conduct bipolar radiofrequency energy between each other.

5. The device of claim 4, wherein the second electrode is configured to ablate tissue with the first electrode, and wherein ablating tissue includes both coagulating and dissecting tissue.

6. The device of claim 1, wherein the plurality of slots are circumferentially disposed about the elongate body.

7. The device of claim 6, wherein the elongate body defines a major longitudinal axis and where each of the plurality of slots is disposed at an angle with respect to the longitudinal axis.

8. The device of claim 7, wherein the plurality of slots provide malleability to the distal portion in all directions.

9. The device of claim 1, further including a handle coupled to the proximal end of the elongate body, and wherein the handle includes a first umbilical in fluid communication with the lumen, the first umbilical being configured to engage a suction device.

10. The device of claim 9, wherein the elongate body defines an opening within the handle distal to its proximal end, the opening being sized to receive the fluid delivery tube.

11. The device of claim 10, wherein the elongate body includes a sealing element disposed within the lumen, wherein the sealing element is configured to prevent aspirated gas from entering the handle.

12. An electrosurgical device, comprising:
an elongate body including a rigid proximal portion, a distal portion, and defining a lumen there though,
a fluid delivery tube disposed within the lumen;
a first electrode coupled to the distal end of the elongate body, the first electrode being configured to ablate tissue with radiofrequency energy;
the distal portion including a plurality of slotted sections circumferentially disposed about the elongate body in fluid communication with the lumen, the plurality of slotted sections being spaced a longitudinal distance proximal from the first electrode, the plurality of slotted sections being configured to aspirate gas released from tissue ablated with the first electrode, the plurality of slotted sections being further configured to provide malleability to the distal portion in all directions; and
the distal portion defining a plurality of unslotted sections, each unslotted section being disposed between adjacent slotted sections, the unslotted sections providing stiffness to the distal portion.

13. The device of claim 12, further including a port in fluid communication with the fluid delivery tube, the port being disposed distal to the plurality of slotted sections and proximal to the first electrode, the port being configured to expel fluid from the fluid delivery tube.

14. The device of claim 13, further include an electrical insulator disposed between the first electrode and the plurality of slotted sections, and wherein the port is disposed within the insulator.

15. The device of claim 14, further including a second electrode adjacent the first electrode, and wherein the electrical insulator is disposed between the first electrode and the second electrode, and the first electrode and the second electrode are configured to conduct bipolar radiofrequency energy between each other.

16. The device of claim 12, wherein the elongate body defines a major longitudinal axis, and wherein each of the plurality of slotted sections is disposed at an angle with respect the major longitudinal axis.

17. The device of claim 12, wherein each slot in the plurality of slotted sections is defines a width between 0.01 inches and 0.03 inches.

18. The device of claim 12, wherein each of the plurality of slotted sections includes between twenty and thirty slots.

19. The device of claim 12, wherein each slot in the plurality of slotted sections is offset from an adjacent one of the plurality of slots by an angle between 10 degrees and 30 degrees.

20. An electrosurgical device, comprising:
an elongate body including a rigid proximal portion, a distal portion, and defining a lumen there though,
a fluid delivery tube disposed within the lumen;
a first electrode and a second electrode coupled to the distal end of the elongate body, the first electrode and the second electrode defining a substantially co-planar surface, the first electrode and the second electrode being configured to ablate tissue with radiofrequency energy;
the distal portion including a plurality of slotted sections circumferentially disposed about the elongate body in fluid communication with the lumen, the plurality of slotted sections being spaced a longitudinal distance proximal from the first electrode, the plurality of slotted sections being configured to aspirate gas released from tissue ablated with the first electrode and the second electrode, the plurality of slotted sections being further configured to provide malleability to the distal portion in all directions, each slot in the plurality of slotted sections defines a width between 0.01 inches and 0.03 inches, each of the plurality of slotted sections includes between twenty and thirty slots, each slot in the plurality of slotted sections is offset from an adjacent one of the plurality of slots by an angle between 10 degrees and 30 degrees; and
the distal portion defining a plurality of unslotted sections, each unslotted section being disposed between adjacent slotted sections, the unslotted sections providing stiffness to the distal portion.
